(19) United States
(12) Patent Application Publication
(10) Pub. No.: US 2022/0087923 A1
(43) Pub. Date: Mar. 24, 2022

(54) ANTIPERSPIRANT AND DEODORANT FORMULATION AND METHOD OF PROCESSING THE SAME

(71) Applicant: Clutch, Inc. d/b/a Carpe, Durham, NC (US)

(72) Inventors: Marc Ward, Millcreek, UT (US); Brice Pettibone, Saratoga Springs, UT (US); McCall Harris, Orem, UT (US); Moe Witwit, Porter Ranch, CA (US); David Spratte, Durham, NC (US); Kasper Kubica, Durham, NC (US)

(21) Appl. No.: 17/468,401

(22) Filed: Sep. 7, 2021

Related U.S. Application Data
(60) Provisional application No. 63/080,427, filed on Sep. 18, 2020.

(51) Int. Cl. A61K 8/96; A61Q 15/00; A61K 8/9794

(52) U.S. Cl. CPC A61K 8/965; A61K 8/9794; A61Q 15/00

(57) ABSTRACT

An antiperspirant and deodorant compositions and method of processing the same is disclosed. the composition may include an aluminum salt as an active ingredient for antiperspirant with activated charcoal powder and a root extract as a natural odor absorbent; a natural fragrance; and an emollient which including at least one of: coconut alkanes and coco-caprylate/carprate. The method includes: adding in sequence according to several defined temperature range, various ingredients in the composition according to a formulation for a defined time duration before cooling to a lower temperature for dispensing.

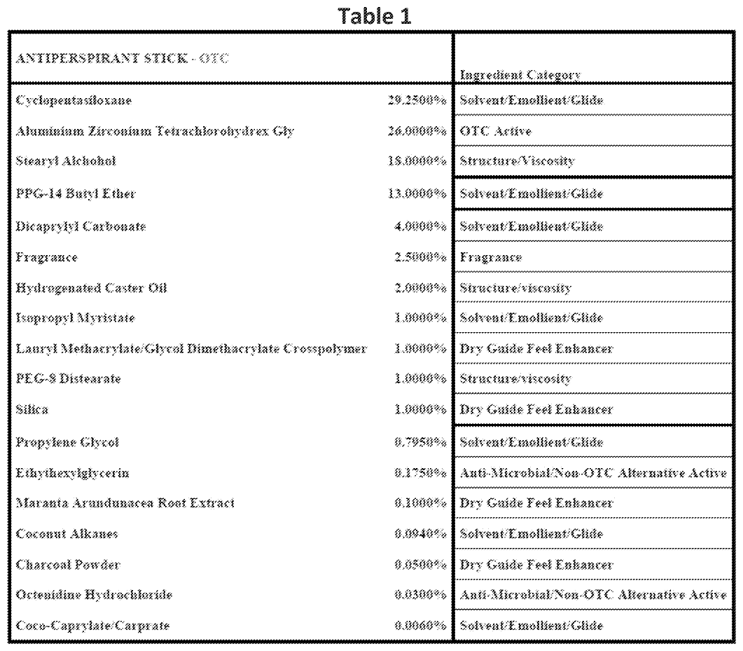

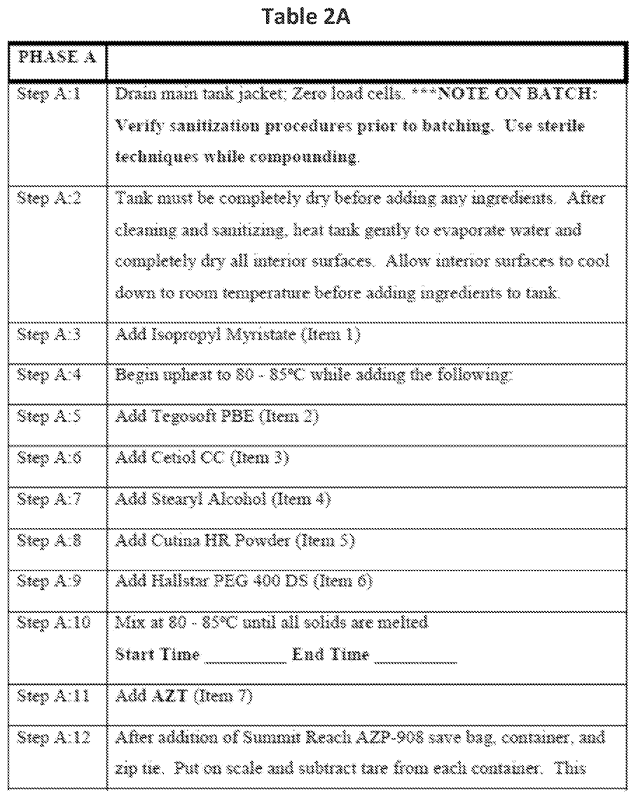

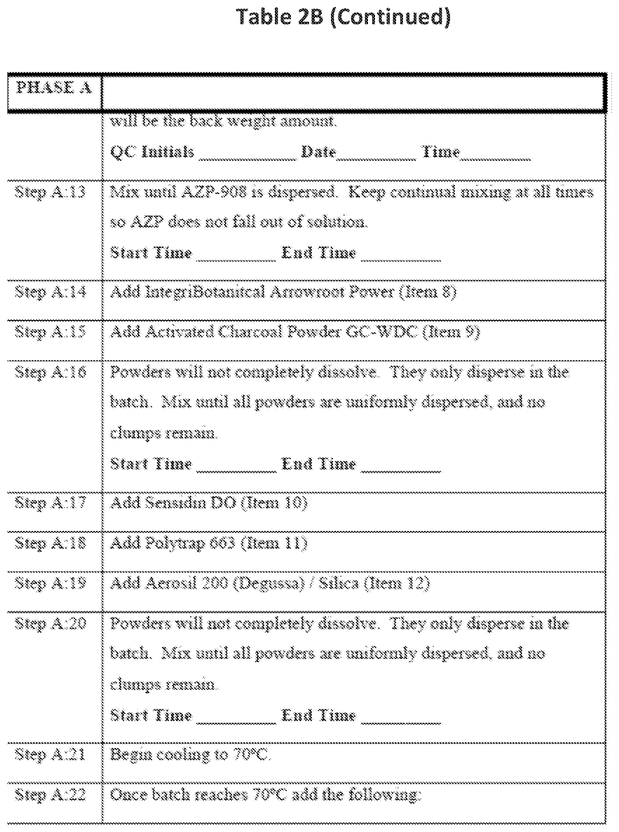

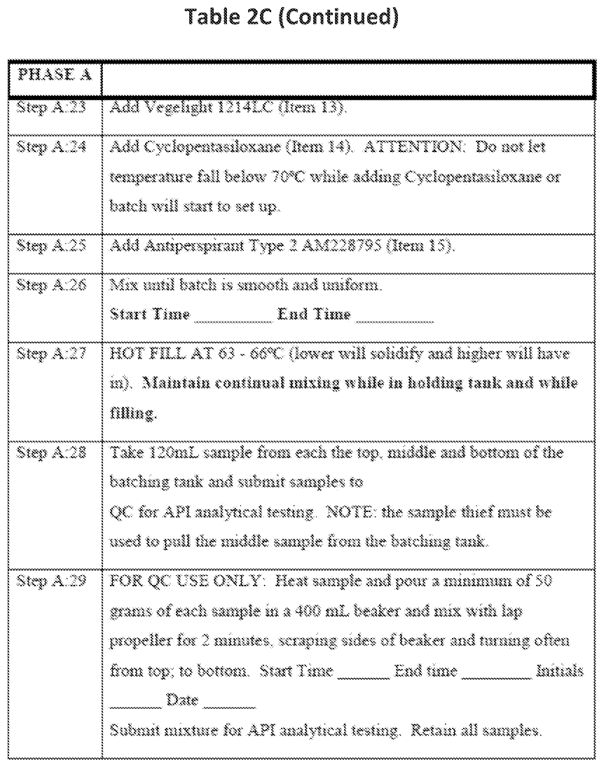

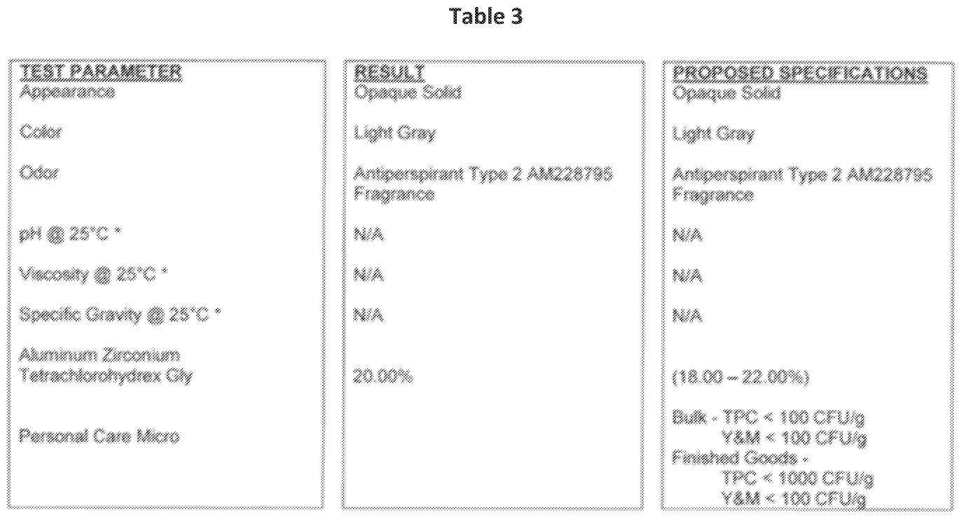

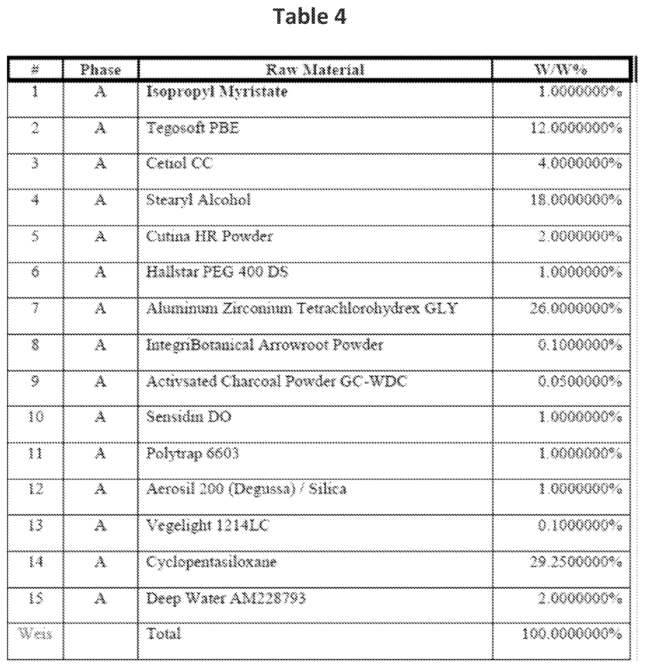

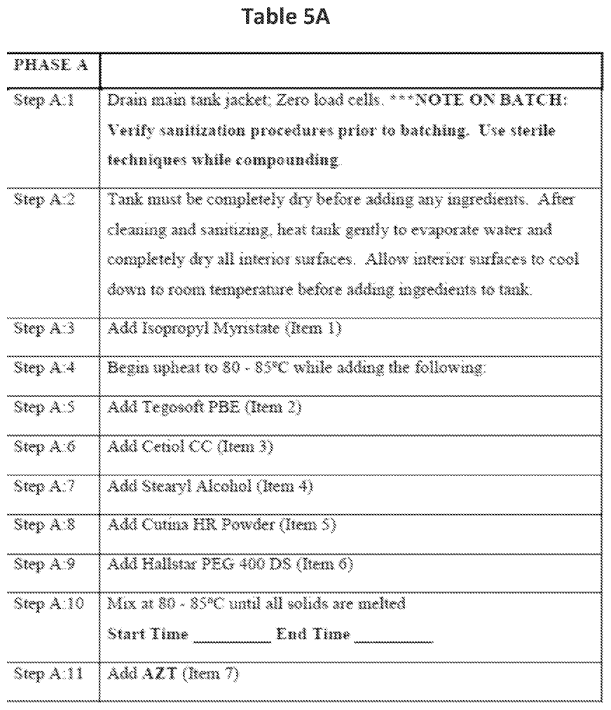

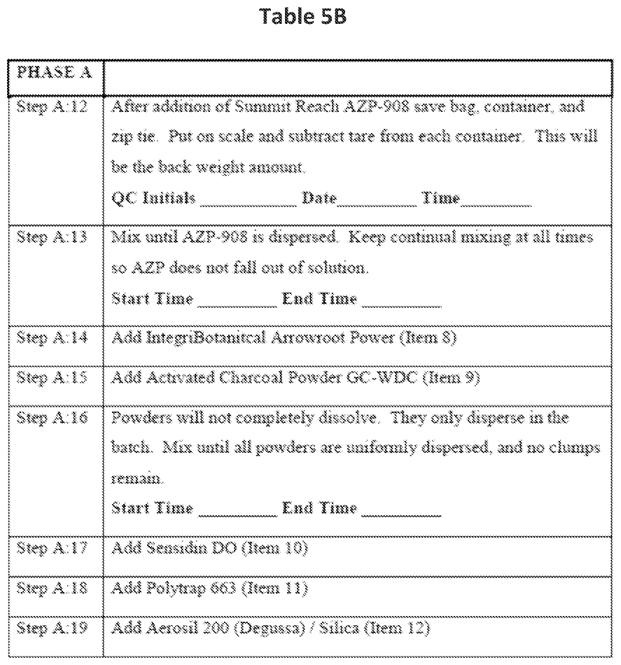

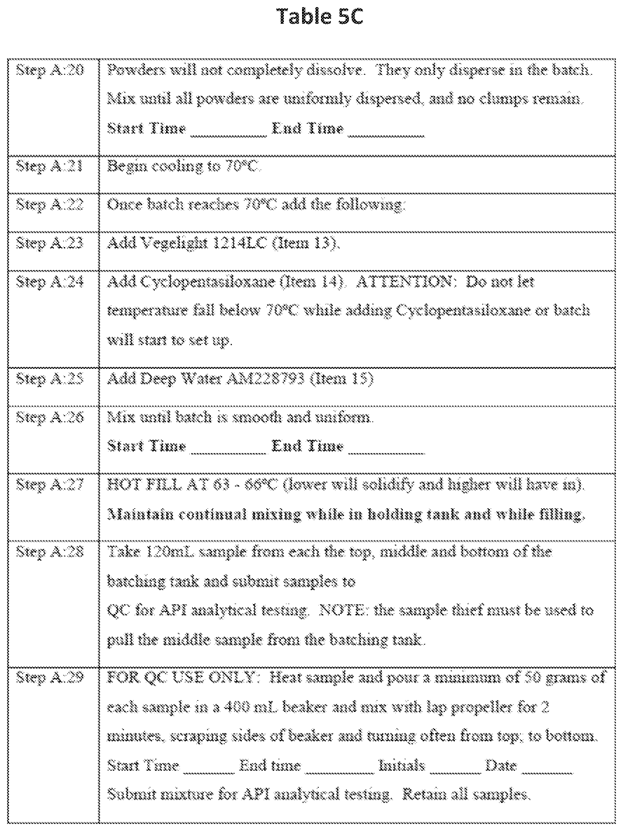

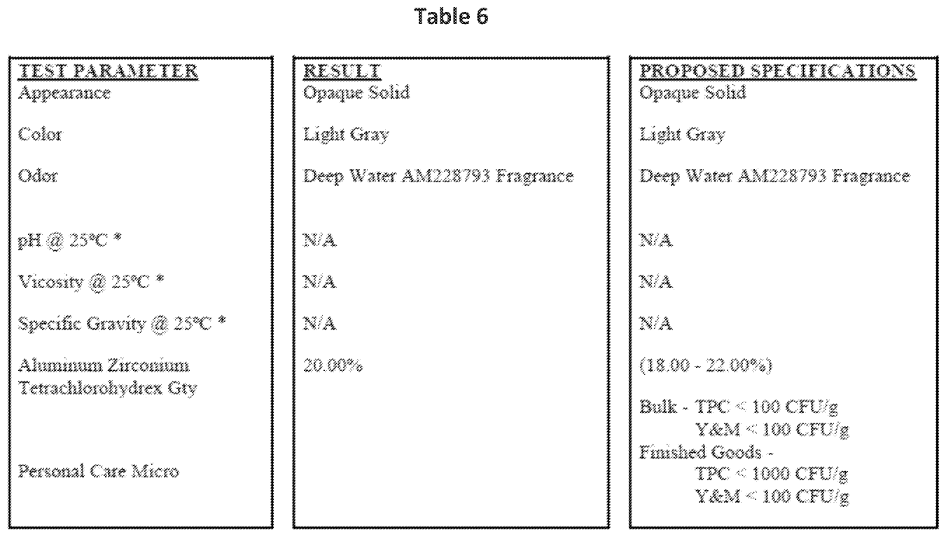

### CROSS REFERENCE TO RELATED APPLICATION

This application claims priority to and the benefit from United States Provisional Patent Application Ser. No. 63/080,427 titled “ANTIPERSPIRANT AND DEODORANT FORMULATION AND METHOD OF PROCESSING THE SAME”, filed on Sep. 18, 2020, the contents of which are herein incorporated by reference in their entirety.

### TECHNICAL FIELD

The present disclosure relates to an antiperspirant and deodorant compositions and method of processing the same. Specifically, a natural odor absorbent is used along with ingredients to provide a smooth feel on the skin in the formulation. Since the odor absorbent and the fragrance in the composition are natural, allergic skin reaction and odor allergy may be minimized to the user and to the surrounding people.

### BACKGROUND

Antiperspirants and deodorants are generally topically applied to body areas having high concentrations of sweat glands to control or limit sweating and odors. Aluminum salt is an active ingredient used in antiperspirants, and when absorbed into the skin, blocks sweat glands areas such as the arm pits, thus effectively keeping these areas relatively dry from wetness due to sweating. Antiperspirants and deodorants formulations also control body odor from the secreted sweat. Nevertheless, sweat from the body itself, moist or dried generally produces odors of varying degrees. In addition, bacteria sometimes may grow in the blocked sweat glands after applying the antiperspirant, may produce unpleasant odors if not properly cleansed. Although an artificial fragrance may be added to the antiperspirant to mask over heavy body odor, nevertheless, a high concentration of the artificial fragrance may act as an air allergen causing allergic reaction to the respiratory tract of the user or to the surrounding people.

### SUMMARY

It would be beneficial to create a deodorant formulation that also prevents or significantly limits perspiration, and which is made from natural, or mostly natural, ingredients.

In an example, an antiperspirant composition, includes at least: an aluminum salt; activated charcoal powder and a root extract; a fragrance; and an emollient, wherein the emollient may include at least one of: coconut alkanes and coco-caprylate/carprate.

In another example, a method for preparing an antiperspirant composition, including the steps of: adding a first emollient that includes isopropyl myristate into a dry mixing tank at room temperature, and heating the first emollient to 80-85° C.; during the heating of the first emollient, adding and completely dissolving a first mixture comprising a second emollient and an emulsifier to the first emollient comprising isopropyl myristate to form a second mixture; adding and uniformly dispersing in a sequence, a known quantity of the following into the second mixture to form a third mixture: aluminum salt, root extract, activated charcoal powder and at least one of: coconut alkanes, coco-caprylate/carprate; and cooling the third mixture to 70 ° C. to maintain for a defined time duration before further cooling to a lower temperature for dispensing.

### BRIEF DESCRIPTION OF THE DRAWINGS

Table 1 illustrates an exemplary list of compositions (ingredients) used in an antiperspirant stick according to an embodiment of the present invention.

Tables 2A-2C illustrate exemplary steps of preparing an antiperspirant according to an embodiment of the present invention.

Table 3 illustrates exemplary test results an antiperspirant according to an embodiment of the present invention.

Table 4 illustrates exemplary compositions (ingredients) by weight in an antiperspirant stick according to an embodiment of the present invention.

Tables 5A-5C illustrate exemplary steps of a batch process for preparing an antiperspirant according to an embodiment of the present invention.

Table 6 illustrates exemplary test results of another antiperspirant.

In one embodiment, a deodorant and antiperspirant composition may include an aluminum salt as an active ingredient for antiperspirant with activated charcoal powder and a root extract as natural odor absorbents; a natural fragrance; and an emollient. The composition may also include at least one of: coconut alkanes and coco-caprylate/carprate.

In another embodiment, moisture absorbing ingredients or dry glide enhancers, such as silica, and POLYTRAP® 6603 Adsorber (Lauryl Methacrylate/Glycol Dimethacrylate Crosspolymer)) may be included to provide excellent overall sweat-reduction and sweat-absorption capability. Additional emollient ingredients and structure/viscosity control ingredients may be added to provide proper smooth texture and odor absorption characteristics.

In another embodiment, a method for preparing the antiperspirant composition with natural deodorizing ingredients is disclosed. The method includes: adding a first emollient including isopropyl myristate into a dry mixing tank at room temperature, and heating the first emollient to 80-85° C. During the heating, adding and completely dissolving a first mixture of emollient and emulsifier to the isopropyl myristate to form a second mixture; adding and uniformly dispersing in a sequence, a known quantity of the following components into the second mixture to form a third mixture: aluminum salt, root extract, activated charcoal powder and at least one of: coconut alkanes, coco-caprylate/carprate. The method further includes cooling the third mixture to 70° C. for a defined time duration before further cooling to a lower temperature for dispensing.

### DETAILED DESCRIPTION

A clear and complete description of the technical solution in the embodiments of the present invention is given below by using the figures thereof; obviously, the described embodiments are only some of the embodiments of the present invention, but not all of the embodiments. Based on the embodiments of the present invention, all other embodiments obtained by ordinary technicians in the art without creative labor shall belong to the scope of protection of the present invention.

Table 1 illustrates an exemplary list of compositions (ingredients) used in an antiperspirant according to an embodiment of the present invention. More specifically, the antiperspirant composition may include an aluminum salt as an active ingredient for antiperspirant with activated charcoal powder and a root extract as natural odor absorbents; a natural fragrance; and an emollient with a composition that includes at least one of: coconut alkanes and coco-caprylate/carprate. In an example, a percentage by weight in composition for the aluminum salt, the activated charcoal powder, the root extract is about 18.0 to about 22.0 percent, about 0.05 percent, and about 0.1 percent, respectively.

In an example, the aluminum salt may include anyone or more of the following compositions by weight: (a) from 0.0% to about 15% aluminum chloride, calculated on the hexahydrate form, in an aqueous solution nonaerosol dosage form, (b) from 0.0% to about 25% aluminum chlorohydrate, (c) aluminum chlorohydrex polyethylene glycol up to 25 percent, (d) aluminum chlorohydrex propylene glycol up to 25 percent, (e) aluminum dichlorohydrate up to 25 percent, (f) aluminum dichlorohydrex polyethylene glycol up to 25 percent, (g) aluminum dichlorohydrex propylene glycol up to 25 percent, (h) aluminum sesquichlorohydrate up to 25 percent, (i) aluminum sesquichlorohydrex polyethylene glycol up to 25 percent, (j) aluminum sesquichlorohydrex propylene glycol up to 25 percent, (k) aluminum zirconium octachlorohydrate up to 20 percent, (l) aluminum zirconium octachlorohydrex glycine up to 20 percent, (m) aluminum zirconium pentachlorohydrate up to 20 percent, (n) aluminum zirconium pentachlorohydrex glycine up to 20 percent, (o) aluminum zirconium tetrachlorohydrate up to 20 percent, (p) aluminum zirconium tetrachlorohydrex glycine 18 to 22 percent, (q) aluminum zirconium trichlorohydrate up to 20 percent, (r) aluminum zirconium trichlorohydrex glycine up to 20 percent, or combinations thereof.

In an example of the antiperspirant, the root extract may include maranta arundinacea root extract, and may include at least a second and a third emollient, the second and third emollients selected from the group consisting of: isopropyl Myristate, cyclopentasiloxane, PPG-14 butyl ether, dicaprylyl carbonate, propylene glycol.

In an example of the antiperspirant may further include lauryl methacrylate/glycol dimethacrylate crosspolymer or silica as a dry glide enhancer. The antiperspirant composition further includes a structural/viscosity composition which includes one or more of: stearyl alcohol, Hydrogenated Castor Oil, PEG-8 distearate, or combinations thereof.

In an example of the antiperspirant may further include an anti-microbial compound comprising: octenidine hydrochloride and ethylhexylglycerin. The antiperspirant composition further includes Tegosoft PBE, Cetiol CC, Cutina HR powder and Hallstar PEG 400DS, wherein the antiperspirant composition may be packaged into a soft solid gel or stick, an aerosol spray, or a liquid.

Tables 2A-2C illustrate exemplary steps of preparing an antiperspirant according to an embodiment of the present invention. In an example, a first batch of the antiperspirant and deodorant composition with a mandarin fragrance was prepared according to the specification and parameters set forth in Tables 1 and 2.

In an example, a method for preparing the antiperspirant composition with natural deodorizing ingredients may include: adding a first emollient including isopropyl myristate into a dry mixing tank at room temperature, and heating the first emollient to 80-85° C. During the heating, adding and completely dissolving a first mixture of emollient and emulsifier to the isopropyl myristate to form a second mixture; adding and uniformly dispersing in a sequence, a known quantity of the following components into the second mixture to form a third mixture: aluminum salt, root extract, activated charcoal powder and at least one of: coconut alkanes, coco-caprylate/carprate. The method further includes cooling the third mixture to 70° C. for a defined time duration before further cooling to a lower temperature for dispensing.

In an example, the method further includes the step of adding and mixing a known quantity of following compositions to the second mixture at 80-85° C. to form a uniform fourth mixture: Sensidin DO, Polytrap 6603 and Aerosil 200 (Degussa)/Silica. The method further includes the step of adding and mixing a known quantity of following compositions to the fourth mixture after cooling to the 70° C. to form a uniform fifth mixture: Vegelight 1214LC, cyclopentasiloxane and deep water.

In an example, the method further includes the step of adding and mixing a known quantity of following compositions to the fourth mixture to form a uniform five mixture at least two of: PPG-14 butyl ether, dicaprylyl carbonate, fragrance, Hydrogenated Castor Oil, lauryl methacrylate/glycol dimethacrylate crosspolymer, PEG-8 distearate, propylene glycol, ethylhexylglycerin, octenidine hydrochloride.

Table 3 illustrates exemplary test results an antiperspirant which contains 20% Aluminum Zirconium Tetrachlorohydrex glycine according to an embodiment of the present invention.

Table 4 illustrates exemplary compositions (ingredients) by weight (e.g., 26% Aluminum Zirconium Tetrachlorohydrex glycine) in an antiperspirant stick according to an embodiment of the present invention. Tables 5A-5C illustrate exemplary steps of a batch process for preparing an antiperspirant according to an embodiment of the present invention. For example, a second batch of the antiperspirant and deodorant composition with a linen fragrance was prepared according to the specification and parameters set forth in Tables 4 and 5.

The second batch was then subject to a set of tests, the results of which are shown in Table 6 (similar to Table 3). Details of the disclosure may be found in the tables and in the recited claims below, which extensive testing data on a large sample of participants who consider themselves “heavy sweaters” may provide favorable responses to the efficacies of the following formulation:
(1) The aluminum zirconium tetrachlorohydrex gly [at max legal concentration of 20%] (active), charcoal powder (inactive), and silica (inactive) as effective moisture control agents
(2) The naturally derived ingredients used: Stearyl Alcohol, Hydrogenated Castor Oil, Maranta Arundinacea Root Extract, Coconut Alkanes, Charcoal Powder, Coco-Caprylate/Caprate
(3) Ingredients responsible for odor control: Aluminum Zirconium Tetrachlorohydrex Gly, 0.5-1.0% Sensidin DO (INCI: Propylene Glycol, Ethylhexylglycerin, Octenidine Hydrochloride).

What have been described above are examples. It is, of course, not possible to describe every conceivable combination of elements, components, or methods, but one of ordinary skill in the art will recognize that many further combinations and permutations are possible. Accordingly, the disclosure is intended to embrace all such alterations, modifications, and variations that fall within the scope of this application, including the appended claims. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, and the term “including” means including but not limited to. The term “based on” means based at least in part on.

1. An antiperspirant composition, comprising:
an aluminum salt;
activated charcoal powder and a root extract;
a fragrance; and
an emollient, the emollient comprising at least one of: coconut alkanes and coco-caprylate/carprate.

2. The antiperspirant composition according to claim 1, wherein a percentage by weight in composition for the aluminum salt, the activated charcoal powder, the root extract is about 18.0 to about 22.0 percent, about 0.05 percent, and about 0.1 percent, respectively.

3. The antiperspirant composition according to claim 2, wherein the aluminum salt comprises one or a combination of:
(a) from 0.0% to about 15% aluminum chloride, calculated on the hexahydrate form, in an aqueous solution nonaerosol dosage form;
(b) from 0.0% to about 25% aluminum chlorohydrate,
(c) aluminum chlorohydrex polyethylene glycol up to 25 percent,
(d) aluminum chlorohydrex propylene glycol up to 25 percent,
(e) aluminum dichlorohydrate up to 25 percent,
(f) aluminum dichlorohydrex polyethylene glycol up to 25 percent,
(g) aluminum dichlorohydrex propylene glycol up to 25 percent,
(h) aluminum sesquichlorohydrate up to 25 percent,
(i) aluminum sesquichlorohydrex polyethylene glycol up to 25 percent,
(j) aluminum sesquichlorohydrex propylene glycol up to 25 percent,
(k) aluminum zirconium octachlorohydrate up to 20 percent,
(l) aluminum zirconium octachlorohydrex glycine up to 20 percent,
(m) aluminum zirconium pentachlorohydrate up to 20 percent,
(n) aluminum zirconium pentachlorohydrex glycine up to 20 percent,
(o) aluminum zirconium tetrachlorohydrate up to 20 percent,
(p) aluminum zirconium tetrachlorohydrex glycine 18 to 22 percent,
(q) aluminum zirconium trichlorohydrate up to 20 percent,
(r) aluminum zirconium trichlorohydrex glycine up to 20 percent, or combinations thereof.

4. The antiperspirant composition according to claim 2, wherein the root extract comprises maranta arundinacea root extract.

5. The antiperspirant composition according to claim 2, further comprising at least a second and a third emollient, the second and third emollients selected from the group consisting of: isopropyl Myristate, cyclopentasiloxane, PPG-14 butyl ether, dicaprylyl carbonate, propylene glycol.

6. The antiperspirant composition according to claim 2, further comprising lauryl methacrylate/glycol dimethacrylate crosspolymer or silica as a dry glide enhancer.

7. The antiperspirant composition according to claim 2, further comprising a structural/viscosity composition comprising one or more of: stearyl alcohol, Hydrogenated Castor Oil, PEG-8 distearate, or combinations thereof.

8. The antiperspirant composition according to claim 2, further comprising an anti-microbial compound comprising: octenidine hydrochloride and ethylhexylglycerin.

9. The antiperspirant composition according to claim 2 further comprising Tegosoft PBE, Cetiol CC, Cutina HR powder and Hallstar PEG 400DS.

10. The antiperspirant composition according to claim 2, wherein the antiperspirant composition a soft solid gel or stick, an aerosol spray, or a liquid.

11. A method for preparing an antiperspirant composition, comprising:
adding a first emollient comprising isopropyl myristate into a dry mixing tank at room temperature, and heating the first emollient to 80-85° C.;
during the heating of the first emollient, adding and completely dissolving a first mixture comprising a second emollient and an emulsifier to the first emollient comprising isopropyl myristate to form a second mixture;
adding and uniformly dispersing in a sequence, a known quantity of the following into the second mixture to form a third mixture: aluminum salt, root extract, activated charcoal powder and at least one of: coconut alkanes, coco-caprylate/carprate; and
cooling the third mixture to 70° C. to maintain for a defined time duration before further cooling to a lower temperature for dispensing.

12. The method according to claim 11, wherein a percentage by weight in composition for the aluminum salt, the activated charcoal powder, the root extract is about 18-22 percent, about 0.05 percent and about 0.1 percent, respectively.

13. The method according to claim 12, wherein the aluminum salt is selected from a group consisting one or a combination of:
(a) aluminum chloride up to 15 percent, calculated on the hexahydrate form, in an aqueous solution nonaerosol dosage form,
(b) aluminum chlorohydrate up to 25 percent,
(c) aluminum chlorohydrex polyethylene glycol up to 25 percent,
(d) aluminum chlorohydrex propylene glycol up to 25 percent,
(e) aluminum dichlorohydrate up to 25 percent,
(f) aluminum dichlorohydrex polyethylene glycol up to 25 percent,
(g) aluminum dichlorohydrex propylene glycol up to 25 percent,
(h) aluminum sesquichlorohydrate up to 25 percent,
(i) aluminum sesquichlorohydrex polyethylene glycol up to 25 percent,
(j) aluminum sesquichlorohydrex propylene glycol up to 25 percent,
(k) aluminum zirconium octachlorohydrate up to 20 percent,
(l) aluminum zirconium octachlorohydrex glycine up to 20 percent,
(m) aluminum zirconium pentachlorohydrate up to 20 percent,
(n) aluminum zirconium pentachlorohydrex glycine up to 20 percent,
(o) aluminum zirconium tetrachlorohydrate up to 20 percent,
(p) aluminum zirconium tetrachlorohydrex glycine 18 to 22 percent,
(q) aluminum zirconium trichlorohydrate up to 20 percent,
(r) aluminum zirconium trichlorohydrex glycine up to 20 percent.

14. The method according to claim 13, wherein the first mixture comprising the second emollient and emulsifier comprises: Tegosoft PBE, Cetiol CC, stearyl alcohol, Cutina HR powder and Hallstar PEG 400DS.

15. The method according to claim 13, wherein the root extract comprises maranta arundinacea root extract.

16. The method according to claim 13, wherein the method further comprises the step of adding and mixing a known quantity of following compositions to the second mixture at 80-85° C. to form a uniform fourth mixture: Sensidin DO, Polytrap 6603 and Aerosil 200 (Degussa)/Silica.

17. The method according to claim 16, further comprising the step of adding and mixing a known quantity of following compositions to the fourth mixture after cooling to the 70° C. to form a uniform fifth mixture: Vegelight 1214LC, cyclopentasiloxane and deep water.

18. The method according to claim 17, the method further comprising the step of adding and mixing a known quantity of following compositions to the fourth mixture to form a uniform five mixture at least two of: PPG-14 butyl ether, dicaprylyl carbonate, fragrance, Hydrogenated Castor Oil, lauryl methacrylate/glycol dimethacrylate crosspolymer, PEG-8 distearate, propylene glycol, ethylhexylglycerin, octenidine hydrochloride.

19. The method according to claim 13, the method further comprising the step dispensing the antiperspirant composition into a physical form comprising one of: soft solid gel or stick, aerosol spray, liquid.
